(19) United States
(12) Patent Application Publication
(10) Pub. No.: US 2026/0325165 A1
(43) Pub. Date: Oct. 22, 2026

(54) SYSTEM AND METHODS FOR ULTRASOUND BASED MEDICAL DEVICE TRACKING

(71) Applicant: Rivanna Medical, Inc., Charlottesville, VA (US)

(72) Inventors: Frank William Mauldin, Troy, VA (US); Adam Dixon, Charlottesville, VA (US); Paul Sheeran, Charlottesville, VA (US); Zachary Leonard, Charlottesville, VA (US); Kevin Owen, Stuart's Draft, VA (US)

(21) Appl. No.: 19/247,487

(22) Filed: Jun. 24, 2025

Related U.S. Application Data
(60) Provisional application No. 63/791,294, filed on Apr. 18, 2025.

(51) Int. Cl. A61B 17/34; A61B 17/00; A61B 34/00; A61B 34/10; A61B 34/20; A61B 90/00

(52) U.S. Cl. CPC A61B 17/3403; A61B 34/10; A61B 34/20; A61B 34/25; A61B 90/08; A61B 2017/00039; A61B 2017/00106; A61B 2017/00154; A61B 2017/00199; A61B 2017/00734; A61B 2017/3405; A61B 2034/102; A61B 2034/107; A61B 2034/2051; A61B 2090/0807

(57) ABSTRACT

A system and method for guiding a needle during percutaneous medical procedures using a magnetic source associated with the needle and magnetic sensors on an ultrasound probe to determine needle tip position, enhance targeting accuracy, and support automated feedback and visualization.

### CROSS-REFERENCE TO RELATED APPLICATION

The present application claims priority to and the benefit of the filing date of U.S. Provisional Application No. 63/791,294, filed Apr. 18, 2025. The disclosures of that application are hereby incorporated by reference herein in their entireties.

### STATEMENT REGARDING FEDERALLY SPONSORED RESEARCH

This invention was made with government support under Grant No. R44AR083337 awarded by the National Institutes of Health (NIH) National Institute of Arthritis and Musculoskeletal and Skin Diseases (NIAMS). The government has certain rights in the invention.

### TECHNICAL FIELD

The present invention is related to an ultrasound-based interventional procedure guidance system and more specifically to an apparatus, system, and method for tracking and guiding medical instruments during ultrasound-based imaging of patient anatomy, including by using one or more magnets.

### BACKGROUND

Ultrasound is increasingly employed to image internal anatomy for both diagnostic purposes and to guide interventional procedures. In particular, ultrasound has proven valuable for procedures involving spinal anatomy—such as lumbar punctures, acute and chronic pain therapies, and focused ultrasound treatments—due to its portability, lack of ionizing radiation, and potential for real-time visualization. Compared to X-ray-based modalities such as computed tomography (CT) and fluoroscopy, which expose patients to ionizing radiation and are impractical for bedside use, ultrasound offers a safer and more accessible alternative. MRI-based approaches, while avoiding ionizing radiation, are similarly constrained by cost, limited accessibility, and incompatibility with the procedural workflows common in emergency and acute care settings.

Despite these advantages, conventional ultrasound systems and probes are designed for general use and are not optimized for spinal anatomy. Their physical geometry often results in suboptimal acoustic windows, obscuring key anatomical landmarks. To overcome these limitations, specialized ultrasound-based multi-array systems have been developed. These systems utilize physically separated and angularly offset transducer arrays that provide superior visualization of spinal structures. Moreover, the spatial separation between arrays allows for the integration of therapeutic tools co-aligned with the imaging plane, enabling real-time or simultaneous image acquisition during interventional procedures.

In parallel with these imaging advancements, significant progress has been made in magnetic tracking technologies for medical device guidance. Magnetically tracked needle assemblies and catheter systems have emerged as powerful tools for enhancing procedural accuracy. These systems typically incorporate a magnetic element—such as a permanent magnet or electromagnet—embedded within or attached to a medical device, such as a needle or catheter. Sensor arrays integrated into the imaging probe or placed on the patient's body detect magnetic field characteristics and compute the position and orientation of the tracked device in three-dimensional space. This positional data can be overlaid with real-time ultrasound images, offering clinicians intuitive visual guidance during device navigation.

Some integrated systems combine ultrasound imaging with magnetic tracking to provide real-time, multi-modal feedback for accurate needle placement during interventional procedures. In such systems, a control console coordinates data from an ultrasound probe and one or more magnetic sensors-either integrated into the probe or placed on the patient's body—that detect the magnetic field generated by a magnet embedded in the needle or associated with the needle, including, by way of example, a needle hub. These sensors determine the needle's position and orientation in three-dimensional space relative to the ultrasound image. This data is used to generate a graphical overlay that enhances the clinician's ability to guide the needle with precision.

This approach is particularly valuable for spine-related procedures, including but not limited to epidural placements, lumbar punctures, paravertebral blocks, erector spinae plane blocks, and sacroiliac joint injections, where accurate needle trajectory and anatomical visualization are critical for safety and efficacy. Similarly, these technologies are also useful for guiding vascular access, joint injections, aspirations, and other percutaneous procedures.

### SUMMARY

Exemplary embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure and the disclosure of which are not limiting in the scope hereof. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention, including as recognized by a skilled artisan.

Embodiments herein overcome limitations of existing ultrasound imaging guidance systems by providing a form factor with multiple arrays, each configured to provide an enhanced acoustic window of the patient's anatomical features, in particular, allowing visualization of elements of the patient anatomy that are difficult or impossible to resolve using conventional approaches, including for example spinal anatomy.

Embodiments herein enable real-time or substantially real-time ultrasound acquisition and medical device tracking during interventional procedures in spinal anatomy, such as percutaneous interventional device guidance procedures facilitated by magnetic-based device tracking technology, as described herein.

Embodiments herein enable multi-angle, multi-array compounding and filtering which can be used to improve the ultrasound imaging visualization of spinal anatomies, vascular anatomies, and inserted medical instruments, such as but not limited to needles or catheters.

Embodiments include a permanent magnetic source embedded in or affixed to a needle assembly, wherein the magnetic source is composed of ferrite, Alnico, or rare-earth materials, or any other suitable magnetic source as recognized by a skilled artisan.

Embodiments include an electromagnetic source embedded in or affixed to a needle assembly, wherein the magnetic source may be, in varying embodiments, battery-operated, sterile, and/or single-use, and configured to be selectively activated or deactivated. In embodiments, the magnetic source, which can be an electromagnet or a permanent magnet, provides user feedback such as vibrations, lights, or sounds indicating operational status or procedural phases.

In embodiments, the ultrasound probe includes an integrated magnetic sensing array configured to detect the three-dimensional (3D) position and orientation of a magnet-embedded or magnet-affixed needle within the sensing volume, wherein the sensing volume is the volume of space in which the magnetic sensing array (such as one or more magnets or one or more magnetic sensors (as described herein), including in some cases one or more magnetic sensors comprising one or magnets) can measure a magnetic source's magnetic field with accuracy sufficient for medical device tracking. The sensing volume can be shaped to align with the needle insertion axis and may be arcuate in configuration to improve spatial tracking performance.

In varying embodiments, the probe can include a central needle guide aligned with the ultrasound imaging plane to ensure consistent in-plane needle visualization. The system can overlay the real-time needle tip position on the ultrasound image and estimate out-of-plane positioning based on needle hub behavior and/or 3D sensor data. In further embodiments, a supplemental visual display may depict a computer-aided design (CAD) model showing the spatial relationship between the needle and probe.

In embodiments, the system provides a procedural guidance interface that includes zone-based proximity indicators, such as a visual countdown from “far” to “close” as the needle approaches the target. These indicators may be shown on-screen and replicated via synchronized light-emitting diodes (LEDs) on the probe to support heads-up workflow during needle maneuvers.

In embodiments, the system includes a pairing mechanism between the one or more magnets (e.g., magnetic element) and the probe, which may be initiated through the user interface or directly on the probe. The system can display pairing status, success confirmation, and error states to guide proper setup.

In embodiments, the system includes bi-directional communication between the probe and the magnetic element or associated components, allowing dynamic feedback at the needle hub or user hand location, including progress indicators or alerts during needle placement.

### BRIEF DESCRIPTION OF THE DRAWINGS

The accompanying drawings illustrate certain aspects of some of the embodiments of the present invention and should not be used to limit or define the invention. Together with the written description the drawings serve to explain certain principles of the invention. For a fuller understanding of the nature and advantages of the present technology, reference is made to the following detailed description of preferred embodiments and in connection with the accompanying drawings.

FIG. 1 depicts an exemplary dual-array probe connected to an exemplary software platform and medical cart, according to embodiments described herein.

FIGS. 2A-2B depict the apparatus guiding a needle with attached magnetic source between an exemplary dual-array probe, according to embodiments described herein.

FIGS. 3A-3B depict an exemplary dual-array probe with an integrated magnetic sensor array, according to embodiments described herein.

FIGS. 4A-4B depict exemplary on-screen indicators during real-time, simultaneous ultrasound imaging and magnetic needle tracking, according to embodiments described herein.

FIG. 5 depicts exemplary patient-attached sterile components that facilitate use of an ultrasound probe during magnetic needle tracking procedures, according to embodiments described herein.

FIG. 6 depicts various stages of a method for tracking and guiding a needle to a desired target within the body of a patient, according to embodiments described herein.

### DETAILED DESCRIPTION

Various embodiments relate to a system and method for facilitating accurate placement of a needle during percutaneous medical procedures using a magnetically tracked needle and real-time or substantially real-time ultrasound imaging. In aspects, the system can employ a magnetic field source integrated with, embedded in, or affixed to, the needle, one or more sensor or one or more sensor arrays on or associated with the ultrasound probe (such as one or more of, Hall effect sensors, magnetoresistive sensors, fluxgate magnetometers, search coil sensors, magneto-impedance sensors, magneto-inductive sensors, and inductive proximity sensors, or other magnetic field sensing technologies familiar to one of ordinary skill in the art), and a control processor configured to determine the three-dimensional (3D) spatial position and orientation of the medical instrument (e.g., needle tip) relative to the ultrasound image plane.

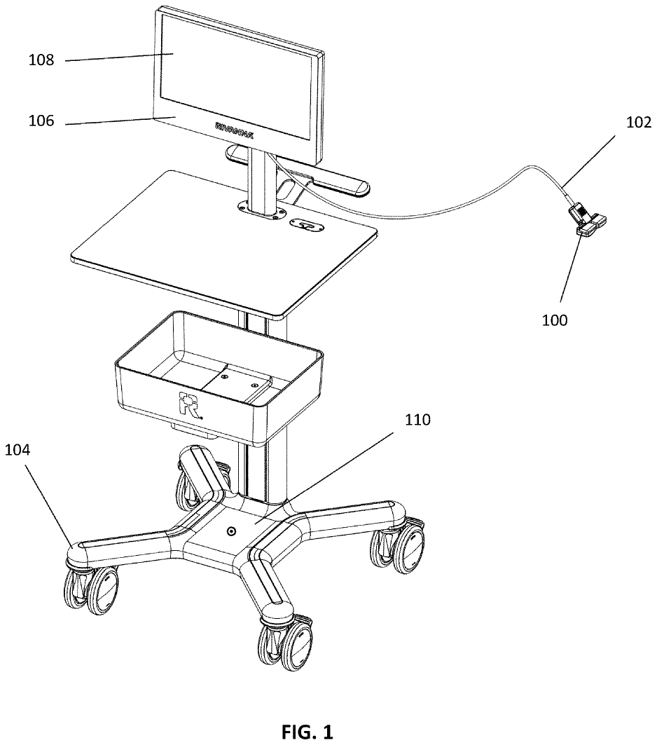

In a non-limiting embodiment depicted in FIG. 1, the ultrasound probe 100 can be connected by an electrical signal cable 102 to a mobile cart 104 to allow the imaging device/system to be moved to the bedside and positioned at the required or desired orientation for acquiring images of the patient's anatomy. The mobile cart 104 can include an enclosure 106 which may contain a computer processor and/or computer controller and a monitor/electronic display 108, battery 110, and other associated electronics familiar to those skilled in the art which are needed to power and communicate with the ultrasound probe 100. In other aspects, the device/system can be powered by another source, such as a wired or hardwired energy providing source (e.g., connected to an electrical outlet). The mobile cart 104 can be outfitted with additional input/output devices such as a keyboard, mouse, or monitor 108, which may also be a touchscreen display. The monitor 108 may be positionally adjustable about the cart in order to orient the ultrasound probe 100 and monitor in various relative positions for the needle tracking and guidance procedure. In a preferred embodiment, the enclosure 106 may simultaneously contain the monitor 108, the computer processor and/or computer controller, and the ultrasound data acquisition electronics. A computer processor and/or computer controller within the enclosure 106 may be used to perform ultrasound signal and image processing steps to form an ultrasound image reconstruction that can be displayed on the monitor 108. Such processing steps are known to those skilled in the art of medical ultrasound and may include but are not limited to: beamforming, bandpass filtering, scan conversion, and image rendering. Two-and three-dimensional images may be rendered using various techniques, including simultaneous display, as described in Mauldin et al., U.S. Pat. No. 11,504,095 (incorporated herein by reference). In a preferred embodiment, the computer processor and/or computer controller in the enclosure 106 can receive signals from the ultrasound probe 100 indicating the position of the probe in the apparatus, which can be used for interpreting spatial position of the real-time image data acquired. In a preferred embodiment, the registration of spatial position of the imaging data can be used to reconstruct three-dimensional ultrasound images that are functionally equivalent or near-equivalent to fluoroscopic or computed tomographic imaging of skeletal anatomy.

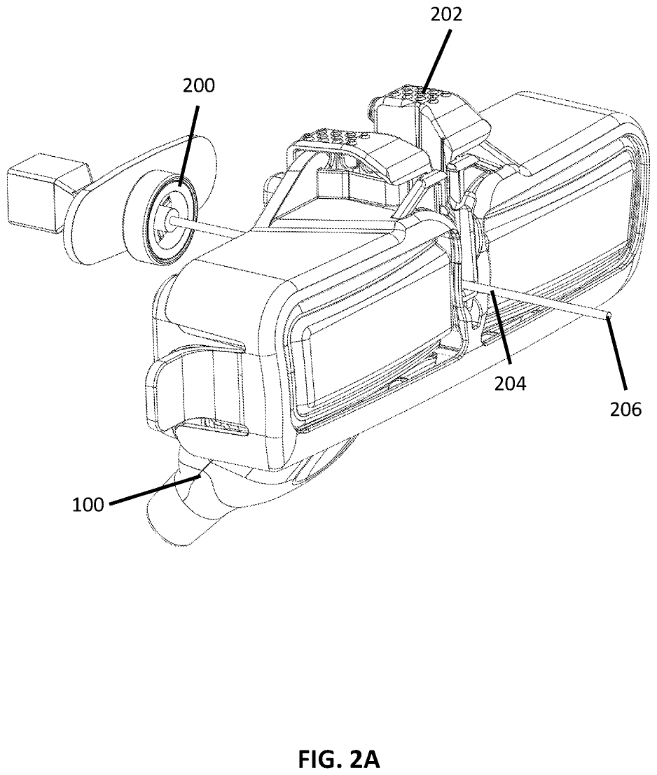

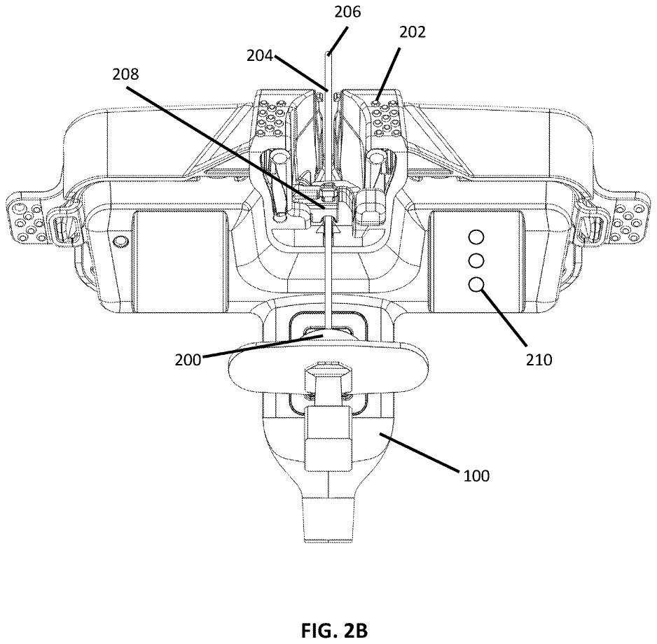

In a non-limiting embodiment depicted in FIG. 2A and FIG. 2B, the needle 204 with affixed magnetic source 200 passes through the needle guide 202. The needle guide 202 (which, in aspects, can be a single-use needle guide) is designed to affix onto an ultrasound probe 100. In the embodiment depicted in FIG. 2A, the ultrasound probe 100 is a dual-array ultrasound probe, but other ultrasound probe configurations may be substituted and are contemplated by the invention disclosure herein. The needle guide 202 has a channel that restricts the insertion trajectory of the needle 204. The channel may be configured to have a clasp 208 that provides for opening and closing of the channel to insert, secure, and remove the needle 206 during a needle placement procedure. In addition, the ultrasound probe 100 can incorporate one or more light emitting diodes 210 or indicators (see, FIG. 2B) (for example, miniature liquid-crystal displays (LCDs)) that relate needle insertion status information to the user.

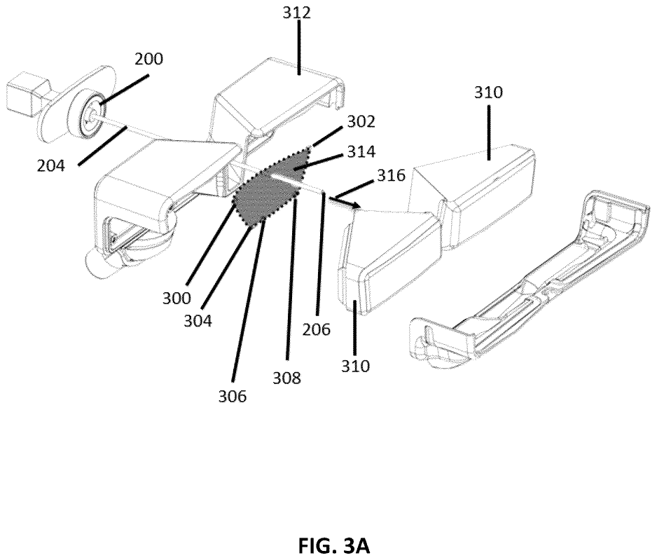

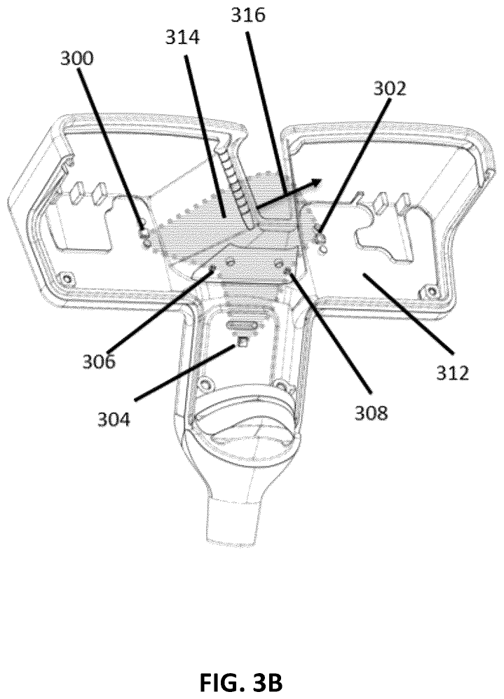

A component-level view of an exemplary dual-array ultrasound probe 100 with embedded magnetic sensors 300, 302, 304, 306, and 308, and ultrasound arrays 310 is depicted in a non-limiting embodiment in FIG. 3A. In another non-limiting embodiment depicted in FIG. 3B, the locations of the embedded magnetic sensors 300, 302, 304, 306, and 308, relative to the housing 312 of the ultrasound probe are depicted. Together, in the depicted embodiment, the multiple magnetic sensors form a magnetic sensor array connected to a computer processor and/or computer controller that is capable of real-time, three-dimensional sensing of magnetic fields in relative proximity to the ultrasound probe 100.

In embodiments, the magnetically tracked needle 204 includes a permanent magnetic source 200 (e.g., a single-use permanent magnetic source) that produces a static magnetic field. (See, FIG. 2A and FIG. 2B.)

In embodiments, the magnetically tracked needle 204 includes a single-use, battery-operated, sterile magnetic source 200 that generates an electromagnetic field. The electromagnetic element or field may be controlled by electronic circuitry or microcontrollers capable of implementing a pulsed operational mode that enables power conservation, specific frequency of electromagnetic emission, and user feedback via timing signals. The needle-affixed electromagnetic source component may include user-facing indicators, such as lights, vibrations, or audible signals, to convey operational status and procedural prompts.

In embodiments, as shown in FIG. 2A and FIG. 2B, the electromagnetic source 200 includes a coil of copper magnet wire powered by an integrated battery, the coil and battery being housed in a unit attachable to, for example, the proximal end of the needle shaft 204. Optionally, the coil of copper magnet wire may be wound around a bobbin or toroid having a high magnetic permeability to strengthen and focus the magnitude of the emitted field. In other embodiments, the emitting element of the electromagnetic source 200 includes a multilayer printed circuit board with traces patterned in circular or looped geometries approximating a coil of copper magnet wire.

In embodiments, the electromagnetic source 200 emits a coded or time-division signal with positive and negative polarity that enables distinction from environmental magnetic noise and improves signal localization.

In embodiments, as shown in FIG. 3A and FIG. 3B, the system includes a multi-element magnetic sensor array 300, 302, 304, 306, and 308, in the ultrasound probe configured to detect the spatial orientation and position of the magnetic source 200 (depicted in FIG. 2A and FIG. 2B). The sensor array can be arranged to provide a 3D sensing volume that encompasses the needle insertion axis. The sensing volume may be arcuate or otherwise configured to maximize the spatial resolution and coverage in regions where the magnetic element is expected to be located with respect to the ultrasound probe and the needle insertion axis. The sensing volume can represent the three-dimensional space, including within the percutaneous region of the patient, detectable using the sensors described herein.

In embodiments, the sterile needle guide 202 component provides a fixed trajectory for needle 204 insertion along a needle insertion axis that is aligned with the ultrasound image plane and an anatomical target.

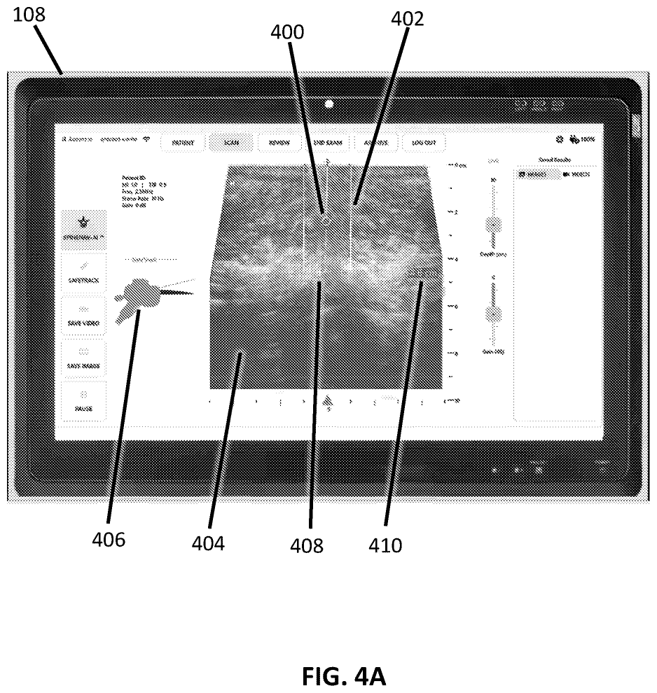

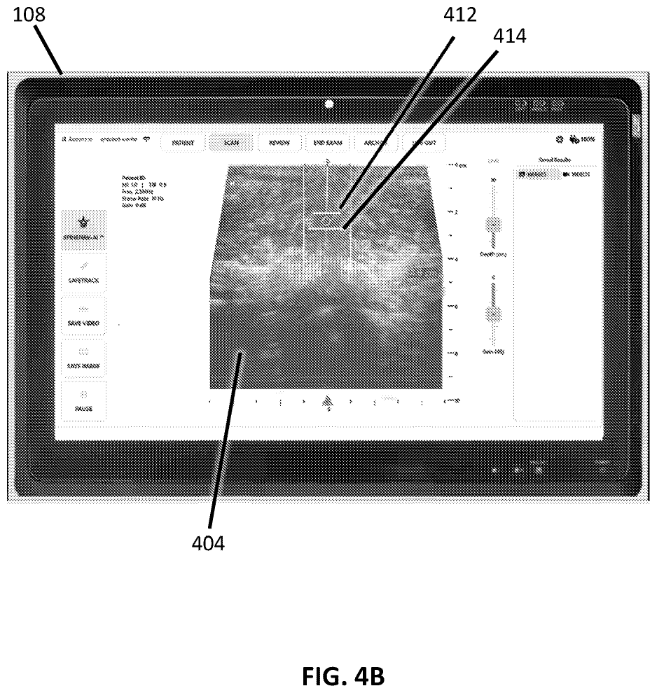

In a non-limiting embodiment shown in FIG. 4A, the system displays on the monitor/electronic display 108 a real-time on-screen indicator 400 of the two-dimensional (2D) projection of the needle tip 206 (see, FIG. 2A and FIG. 2B) and the needle trajectory guide lanes 402, overlaid on the ultrasound image 404. Additionally, a calculated estimate of the needle's out-of-plane position 406 can be presented using a rendered CAD model or iconography that illustrates the spatial relationship between the needle and the probe, facilitating intuitive navigation. Furthermore, the system may optionally depict indicators relating anatomical target positions 408 for needle insertion, the distance to which may be indicated on-screen via measurement indicators 410.

In embodiments, the system displays proximity-based needle trajectory guidance lanes 402 with a zone-based countdown that indicates needle position relative to the anatomical target 408. These zones (e.g., “far,” “closer,” “close”) assist the user in understanding spatial proximity of the needle tip 206 to sensitive targets. The zone indicators may be visualized both via on-screen indicators 402 and via synchronized LEDs embedded in the probe housing 210, allowing the user to maintain visual focus on the procedural field rather than the display.

In embodiments, the system is configured to automatically detect anatomical targets 408 in the ultrasound image and estimate and display the depth to such targets 410. The system may use this estimated distance as the reference point(s) for countdown indicators 402 and related visual, haptic, or auditory guidance. Automatic detection of anatomical targets 408 may be achieved via template matching algorithms, artificial intelligence algorithms, machine learning algorithms, among others. This target detection capability improves standardization, reduces variability in operator-defined targets, and enhances tracking accuracy.

In a non-limiting example shown in FIG. 4B, a calculated estimate of the needle's out-of-plane position may be represented by graphical indicators along the needle trajectory that indicate the relative orientation of the needle via a proximal indicator 412 and a distal indicator 414. In aspects, when the indicators are matched in appearance, such as in size, intensity, hue, saturation, luminance, or other properties that would be understood by one of ordinary skill in the art, the needle is considered to be in-plane. FIG. 4B shows a representation of an out-of-plane needle position where the distal tip of the needle is forward of the probe housing, resulting in a smaller proximal indicator and a larger distal indicator.

In embodiments, the ultrasound probe includes on-probe indicators 210 (see, FIG. 2A and FIG. 2b) such as LEDs, which can synchronize with on-screen graphics to provide users with real-time procedural guidance. These indicators can convey relative needle-to-target distance, current system status and/or progress toward a series of procedural tasks, or out-of-plane deviations without requiring the user to shift focus away from the ultrasound probe 100 or patient.

In embodiments, a pairing mechanism between the probe 100 and magnetic element 200 ensures proper initialization of the system. The pairing process may include both on-probe indicators 210 (see, FIG. 2B) and on-screen feedback, such as indicating graphics 406 (see, FIG. 4A), including but not limited to error messages and confirmation indicators. This can help prevent procedural delays or inaccuracies due to pairing failures.

In embodiments, bidirectional communication is established between the ultrasound probe 100 and the magnetic element 200. In aspects, this can enable control signals calculated by a first processor and/or controller (in aspects, within or operatively connected to the ultrasound probe 100 or system enclosure 106), to be transmitted to a second processor and/or controller (in aspects, within or operatively connected to the magnetic element 200), in an example. These control signals are received by the second processor and/or controller located within or operatively connected to the magnetic element 200, which is affixed to the needle or needle assembly. In response, the second processor and/or controller activates feedback mechanisms—such as lights, vibrations, or tones—issued from the magnetic element (or the second processor and/or controller) near the user's hand position on the needle or needle assembly, to indicate procedural status or transitions in tracking or guidance.

In embodiments, the system allows for active control and feedback of the magnetic source. According to the present invention, the magnetic source 200 can be synchronized with one or more magnetic sensors or one or more magnet sensor arrays for improved accuracy and reduced sensor noise.

In embodiments of the present invention, a 3D magnetic sensor array can support accurate out-of-plane tracking to assist with anatomically complex or deep procedures like spine access.

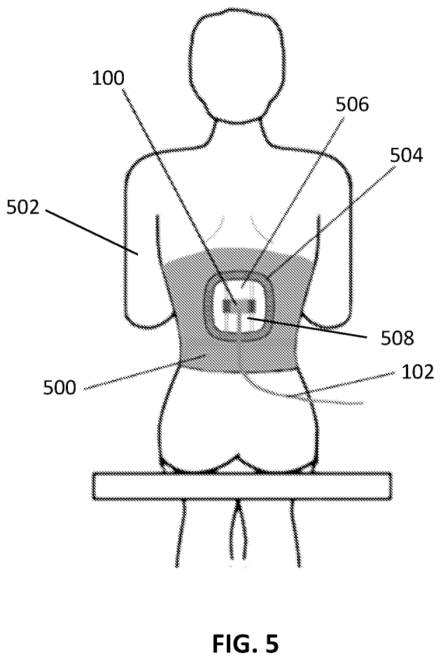

In a non-limiting embodiment illustrated in FIG. 5, a sterile drape 500 may be placed over the patient or a portion of the patient 502 prior to the procedure. The drape can include an adhesive frame element 504 attached to the patient. This frame can surround a fenestration 506 in the drape, allowing access to the patient's skin. The ultrasound probe 100 containing, in aspects, the magnetic sensors (300, 302, 304, 306, and 308 (see, FIG. 3A and FIG. 3B)) can be temporarily affixed to the patient anatomy by straps or bands 508 attached to the frame element 504 to aid in procedure guidance.

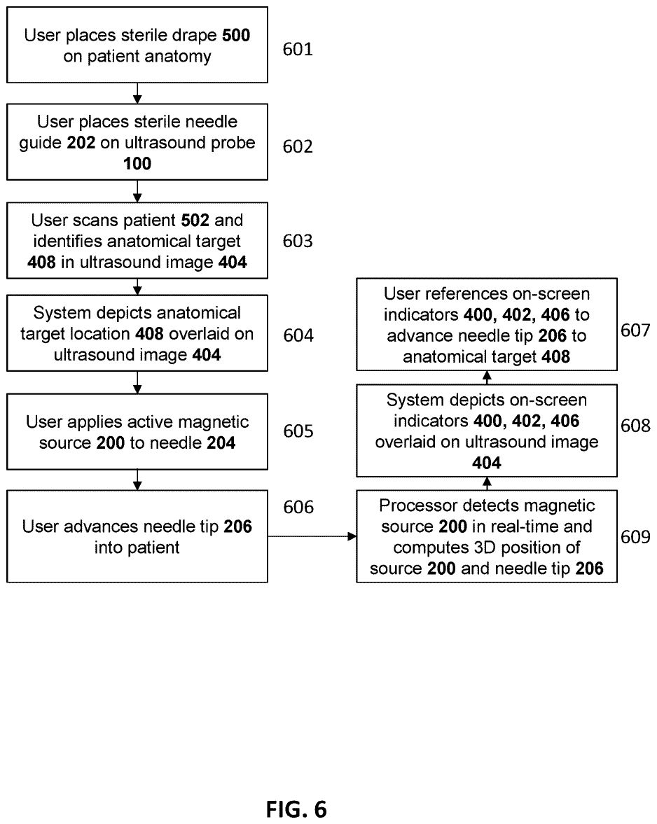

In another non-limiting embodiment depicted in FIG. 6, the system and method comprise any number of steps in the process to enable the current invention. In aspects, a user places a sterile drape over or on a patient anatomy on or near the site for the needle insertion 601. In aspects, the user places a needle guide, preferably a sterile needle guide, on an ultrasound probe, as described herein 602. The user ultrasound scans the patient and identifies the anatomical target for needle tip insertion in an ultrasound image from the ultrasound probe 603. The system depicts the target anatomical location overlaid on the ultrasound image 604. Before insertion, the user applies an active magnetic source to the needle or needle assembly, and the user puts the needle in a guide channel associated with the ultrasound probe 605. The magnetic source of the needle or needle assembly is detected by one or more magnetic sensors or one or more magnetic sensor arrays in, on, or operatively connected to, the ultrasound probe. The user will then advance the needle tip into the patient and towards the target anatomy, the system sensing, using magnets, a location of the needle and/or needle tip 606. A processor and/or controller detects the magnetic source in real-time or substantially real-time and computes a 3D position of the magnetic source and the associated needle and/or needle tip 607. On a monitor/electronic display, the system can depict on-screen indicators overlaid on the ultrasound image 608. This allows a user to reference the on-screen indicators to advance the needle tip to the anatomical target to assist with needle guidance into the patient and towards and into the anatomical target location 609.

In embodiments, the system integrates multi-angle, multi-array 310, e.g. FIG. 3A, ultrasound acquisition and compounding techniques to enhance image quality, particularly for complex anatomical areas such as the spine, vasculature, or soft tissue interfaces. This allows for improved visualization of anatomical targets and inserted instruments.

As would be understood by one or ordinary skill in the art, a needle assembly can include any one or more of the following:

Needle: A hollow, sharp (usually metal) tube that penetrates the skin, anatomy, target anatomy, or patient anatomical location;

Hub: A base (usually plastic or metal) that connects the needle to a syringe or other device(s);

Luer lock or slip tip: A connection mechanism on the hub that attaches to syringes or other medical devices;

Bevel: An angled tip of the needle that creates a sharp point for easier insertion;

Cannula: A hollow shaft of the needle through which, for example, fluids pass;

Safety shield or cap: A protective cover that prevents accidental needle sticks when not in use;

Gauge: Though not a physical part, it can refer to the needle's diameter and can be marked on a needle assembly;

Shaft: A main body of the needle between the hub and the bevel;

Flashback chamber: In some needle assemblies, especially those used for blood collection, there can be a clear chamber that allows visualization of blood flow (or other fluid) to confirm proper placement, such as in a vein;

Needle guard: In some designs, a retractable or sliding guard can cover the needle after use for safety purposes.

Embodiments of the invention also include a computer-readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general-purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specifications and examples be considered as exemplary in nature and that variations that do not depart from the essence of the invention fall within the scope of the invention. Further, all references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figures may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

1. A system for tracking or guiding a needle during a percutaneous medical procedure, the system comprising:
a magnetic source operatively connected to, located within, or located on, the needle or a needle assembly comprising the needle and a hub;
an ultrasound probe comprising a probe housing and at least one ultrasound transducer array operative to acquire one or more ultrasound images of a target anatomy or target anatomical location and define an ultrasound image plane;
the ultrasound probe further comprising at least three magnetic sensors, the at least three magnetic sensors located at known physical positions within or on the probe housing in a non-collinear three-dimensional spatial arrangement and configured to sense the magnetic source's magnetic field;
wherein the known physical positions of the at least three magnetic sensors geometrically surround and define a needle insertion region allowing for a plurality of possible needle insertion trajectories, angles, or axes, such that a three-dimensional magnetic sensing volume is determined by the known physical positions of the at least three magnetic sensors;
wherein at least one of the plurality of possible needle insertion trajectories, angles, or axes intersects both the needle insertion region and the ultrasound image plane;
a monitor or electronic display configured to show the one or more ultrasound images; and
a processor, in response to executable instructions, operative to:
receive magnetic field information from the at least three magnetic sensors;
determine a three-dimensional spatial location and orientation of the magnetic source relative to the known physical positions of the at least three magnetic sensors;
track a spatial location and orientation of the needle as the needle is inserted and/or guided towards the target anatomy or target anatomical location;
generate a visual representation of the needle spatial location and orientation and combine that visual representation with the one or more ultrasound images, by overlaying the visual representation with the one or more ultrasound images or overlaying the one or more ultrasound images with the visual representation; and
determine a deviation of the needle relative to the ultrasound image plane based on the tracked spatial location and orientation of the needle, and display an out-of-plane deviation indicator based on the determined deviation.

2. The system of claim 1, wherein the magnetic source is provided as a sterile, disposable unit.

3. The system of claim 1, wherein the magnetic source is an electromagnet.

4. (canceled)

5. The system of claim 1, wherein the at least three magnetic sensors are mounted at the known physical positions within or on the probe housing to define the three-dimensional magnetic sensing volume that includes a trajectory, an angle, or an axis, of needle insertion.

6. The system of claim 5, wherein the magnetic source is an electromagnet, wherein the electromagnet is pulsed, and wherein the at least three magnetic sensors are configured to sense the pulsed magnetic source.

7. The system of claim 1, further comprising a needle guide removably attachable to the ultrasound probe and configured to align the needle with the ultrasound image plane.

8. The system of claim 1, wherein the ultrasound probe comprises two ultrasound transducer arrays and a removably attachable needle guide located between the two ultrasound transducer arrays, the needle guide defining a physical gap providing or allowing for the plurality of possible needle insertion trajectories, angles, or axes, for insertion, guidance, tracking, or combinations thereof, of the needle, and wherein the physical gap is positioned within overlapping fields of view of the two ultrasound transducer arrays so that at least one of the plurality of possible needle insertion trajectories, angles, or axes, is located substantially within the ultrasound image plane at a needle insertion site and remains substantially within the ultrasound image plane during needle insertion, tracking, guidance, or combinations thereof.

9. The system of claim 1, wherein the processor configured with instructions is further operative to display on the monitor or electronic display indicators that show or alert a user to the out-of-plane deviation(s) of the needle.

10. The system of claim 9, wherein the out-of-plane deviation(s) of the needle are indicated by a two-dimensional or three-dimensional model representation of the needle displayed on the monitor or electronic display.

11. The system of claim 9, wherein the out-of-plane deviation(s) of the needle are indicated by graphical overlays on a B-mode image along a needle trajectory.

12. The system of claim 1, wherein the processor configured with instructions is further operative to display real-time progress of the needle towards the target anatomy or target anatomical location, including visual zones representing a distance to the target anatomy or target anatomical location.

13. The system of claim 1, wherein the ultrasound probe further comprises on-probe visual indicators synchronized with graphics displayed on the monitor or electronic display.

14. The system of claim 1, wherein the processor configured with instructions is further operative to identify a location of the target anatomy or target anatomical location and visually depict its location and distance from the needle on the monitor or electronic display.

15. The system of claim 14, wherein the visual depiction of the needle is altered, including by one or more of, adjusting an intensity, hue, saturation, or luminance, of the visual depiction, based on the distance between (a) the location of the target anatomy or target anatomical location and (b) the needle or needle tip.

16. The system of claim 14, wherein the processor configured with instructions is further operative to generate one or more alternative, virtual representations of the needle to assist a user with tracking and guidance of the needle to the target anatomy or target anatomical location, and wherein the visual depiction of the needle is altered by selecting between the generated one or more alternative, virtual representations of the needle based on the distance between (a) the target anatomy or target anatomical location and (b) the needle or needle tip.

17. A system for tracking or guiding a needle during a percutaneous medical procedure, the system comprising:
a magnetic source associated with the needle, the magnetic source generating a magnetic field;
an ultrasound probe including a probe housing and at least one ultrasound transducer array, the ultrasound probe operative to acquire a plurality of ultrasound images of an anatomical target and define an ultrasound image plane;
the ultrasound probe further including at least three magnetic sensors located at known physical positions within or on the probe housing in a non-collinear three-dimensional spatial arrangement and configured to sense the magnetic field;
wherein the known physical positions of the at least three magnetic sensors geometrically surround and define a needle insertion region allowing for a plurality of possible needle insertion trajectories, angles, or axes, such that a magnetic three-dimensional sensing volume is determined by the known physical positions of the at least three magnetic sensors;
wherein at least one of the plurality of possible needle insertion trajectories, angles, or axes intersects both the needle insertion region and the ultrasound image plane;
an output display device operative to display the plurality of ultrasound images acquired by the ultrasound probe; and
at least one processing device, in response to executable instructions, operative to:
receive magnetic field information from the at least three magnetic sensors;
determine a three-dimensional spatial location and orientation of the magnetic source relative to the known physical positions of the at least three magnetic sensors;
track a spatial location and orientation of the needle as the needle is inserted and/or guided towards the anatomical target;
generate a visual representation of the needle spatial location and orientation in combination with at least one of the plurality of ultrasound images as visible on the output display device; and
determine a deviation of the needle relative to the ultrasound image plane based on the tracked spatial location and orientation of the needle, and display an out-of-plane deviation indicator based on the determined deviation.

18. The system of claim 17, wherein the magnetic source is an electromagnet.

19. The system of claim 18, wherein the electromagnet is powered by a battery.

20. The system of claim 17, wherein the at least three magnetic sensors are mounted at the known physical positions within or on the probe housing to define the three-dimensional magnetic sensing volume that includes an axis, trajectory, angle, or location, of needle insertion.

21. (canceled)

22. (canceled)

23. (canceled)

24. (canceled)

25. (canceled)

26. (canceled)

27. (canceled)

28. (canceled)

29. (canceled)

30. (canceled)

31. The system of claim 8, wherein the needle insertion region is at least partially overlapping with the physical gap between the two ultrasound transducer arrays.
